(19) United States
(12) Patent Application Publication
(10) Pub. No.: US 2023/0263430 A1
(43) Pub. Date: Aug. 24, 2023

(54) CLOSED-LOOP ARTIFICIAL PANCREAS INSULIN INFUSION CONTROL SYSTEM

(71) Applicant: MEDTRUM TECHNOLOGIES INC., Shanghai (CN)

(72) Inventor: Cuijun YANG, Shanghai (CN)

(73) Assignee: MEDTRUM TECHNOLOGIES INC., Shanghai (CN)

(21) Appl. No.: 18/022,524

(22) PCT Filed: Aug. 26, 2020
(86) PCT No.: PCT/CN2020/111169

§ 371 Date: Feb. 22, 2023

(51) Int. Cl. A61B 5/11; G16H 10/60

(52) U.S. Cl. CPC A61B 5/1118; G16H 10/60

(57) ABSTRACT

A closed-loop artificial pancreas insulin infusion control system, includes: a detection module; a program module is configured to obtain the insulin dose infused per day by users; an infusion module, connected to and controlled by the program module, is configured to infuse insulin required according to the data of the current insulin infusion dose; and a motion sensor configured to sense the user's physical activity status which is sent to the program module and is one of the variable factors of the total daily dose (TDD) algorithm or the current insulin infusion algorithm. This system automatically detect the physical condition of the user and accurately calculate the TDD value and the current insulin infusion dose, enhancing user experience.

### TECHNICAL FIELD

The present invention mainly relates to the field of medical device, and in particular, to a closed-loop artificial pancreas insulin infusion control system.

### BACKGROUND

The pancreas in a normal person can automatically monitor the amount of glucose in the blood and automatically secrete the required dosage of insulin/glucagon. However, for diabetic patients, the function of the pancreas is abnormal, and the pancreas cannot normally secrete required dosage of insulin. Therefore, diabetes is a metabolic disease caused by abnormal pancreatic function and also a lifelong disease. At present, medical technology cannot cure diabetes, but can only control the onset and development of diabetes and its complications by stabilizing blood glucose.

Patients with diabetes need to check their blood glucose before injecting insulin into the body. At present, most of the detection methods can continuously detect blood glucose, and send the blood glucose data to the remote device in real time for the user to view. This detection method is called Continuous Glucose Monitoring (CGM), which requires the detection device to be attached to the surface of the patients' skin, and the sensor carried by the device is inserted into the subcutaneous tissue fluid for testing. According to the blood glucose (BG) level, the infusion device, as a closed-loop or semi-closed-loop artificial pancreas, injects the currently required insulin dose.

At present, the detection device and the infusion device are connected to each other to form a closed-loop artificial pancreas with the processing of the program module. While the program module is calculating the insulin infusion dose, total daily dose (TDD) is an important parameter with many determinants, such as physical conditions, physiological conditions, etc.

However, the closed-loop artificial pancreas in prior art needs to be manually input the physical conditions instead of automatically detecting, and the TDD value cannot be accurately obtained, resulting in inaccurate current insulin infusion dose and worsening user experience.

Therefore, in the prior art, there is an urgent need for a closed-loop artificial pancreas insulin infusion control system that can automatically detect the physical condition and accurately calculate the current infusion dose.

### BRIEF SUMMARY OF THE INVENTION

The embodiment of the present invention discloses a closed-loop artificial pancreas insulin infusion control system which can automatically detect the physical condition of the user and accurately calculate the TDD value and the current insulin infusion dose, enhancing user experience.

The invention discloses a closed-loop artificial pancreas insulin infusion control system, including: a detection module configured to detect blood glucose; a program module, connected to the detection module, is configured to obtain the insulin dose infused per day by users, and is also imported into the total daily dose algorithm and the current insulin infusion algorithm, wherein, according to the insulin dose infused per day by users, the total daily dose algorithm is used to calculate the total daily dose; according to the blood glucose detected, the insulin dose infused per day by users or total daily dose, the current insulin infusion algorithm is used to calculate the current insulin infusion dose; an infusion module, connected to and controlled by the program module, is configured to infuse insulin required according to the data of the current insulin infusion dose; and a motion sensor configured to automatically sense the user's physical activity status which can be sent to the program module and is one of the variable factors of the total daily dose algorithm or the current insulin infusion algorithm.

According to one aspect of the present invention, the program module includes a manual input interface or an automatic detection sub-module, and the method for the program module to obtain the insulin dose infused per day by users includes: the insulin dose infused per day by users is manually input into the program module through the manual input interface; or the insulin dose infused per day by users is automatically detected, stored and calculated by the automatic detection sub-module.

According to one aspect of the present invention, the insulin dose infused per day by users includes the total amount of daily infusion dose data, or the bolus and basal data infused in different time periods, or the temporary basal data and the correction bolus data, or the infusion data after different events.

According to one aspect of the present invention, the total daily dose is obtained by calculating the total amount of daily infusion dose data in the previous two or more days according to the total daily dose algorithm, and the total daily dose is the average or median of the insulin dose infused per day by users, and the total daily dose is one variable factor of the current insulin infusion algorithm.

According to one aspect of the present invention, the average includes an arithmetic average or a weighted average.

According to one aspect of the present invention, the variable factors of the total daily dose algorithm include one or more of the user's physical activity status, physiological status, psychological status, and meal status.

According to one aspect of the present invention, the physiological status includes one or more factors of weight, gender, age, disease, and menstrual period.

According to one aspect of the present invention, the physical activity status includes general body stretching, exercise, or sleep.

According to one aspect of the present invention, the meal information, exercise information, sleep information, or physical condition information can be manually input into the program module through the manual input interface.

According to one aspect of the present invention, the motion sensor is provided in the detection module, the program module or the infusion module.

According to one aspect of the present invention, the motion sensor includes a three-axis acceleration sensor or a gyroscope.

According to one aspect of the present invention, any two of the detection module, the program module and the infusion module are connected to each other configured to form a single structure whose attached position on the shin is different from the third module.

According to one aspect of the present invention, the detection module, the program module and the infusion module are connected together configured to form a single structure which is attached on only one position on the skin.

Compared with the prior art, the technical solution of the present invention has the following advantages:

In the closed-loop artificial pancreas insulin infusion control system disclosed in the present invention, the detection module is used to detect blood glucose; a program module, connected to the detection module, is configured to obtain the insulin dose infused per day by users, and is also imported into the total daily dose algorithm and the current insulin infusion algorithm, wherein, according to the insulin dose infused per day by users, the total daily dose algorithm is used to calculate the total daily dose; according to the blood glucose detected, insulin dose infused per day by users or total daily dose, the current insulin infusion algorithm is used to calculate the current insulin infusion dose. The program module is imported into the total daily dose algorithm and the current insulin infusion algorithm. Using the detection data, the insulin dose infused per day by users and the total daily dose alone or in combination makes the current insulin infusion dose more accurate. Secondly, the control system also includes a motion sensor configured to automatically sense the user's physical activity which can be sent to the program module and is one of the variable factors of the total daily dose algorithm or the current insulin infusion algorithm. Compared with manually inputting, the motion sensor can automatically and accurately sense the user's physical activity status which can be sent to the program module, improving the accuracy of the calculation of the total daily dose or the current insulin infusion dose, and enhancing the user experience.

Furthermore, the program module includes a manual input interface or an automatic detection sub-module, and the method for the program module to obtain the insulin dose infused per day by users includes: the insulin dose infused per day by users is manually input into the program module through the manual input interface; or the insulin dose infused per day by users is automatically detected, stored and calculated by the automatic detection sub-module. The manual input interface or the automatic detection sub-module can be used alone or a combined, which enhances the flexibility using the device. Secondly, with the manual input interface and the automatic detection sub-module used in combination, the data automatically detected and manually input can be combined and compared to make the program module adjust the algorithm in real time, helping to make the calculation result more accurate.

Furthermore, the physical activity status includes general body stretching, exercise or sleep. The control system can distinguish normal activities, exercise and sleep, making the control system more refined to control blood glucose level.

Furthermore, the motion sensor is provided in the detection module, the program module or the infusion module. The motion sensor provided in the control system, not disposed in other structure, can improve the integration of the control system as much as possible, reduce the size of the device, and enhance the user experience.

Furthermore, the motion sensor includes a three-axis acceleration sensor or a gyroscope. The three-axis acceleration sensor or gyroscope can sense the body's activity intensity, activity mode or body posture accurately, ultimately improving the accuracy of the calculation result of the infusion dose.

Furthermore, the detection module, the program module and the infusion module are connected together configured to form a single structure which is attached on only one position on the skin. If the three modules are connected as a whole and attached in the only one position, the number of the device on the user skin will be reduced, thereby reducing the interference of more attached devices on user activities. At the same time, it also effectively solves the problem of the poor wireless communication between separating devices, further enhancing the user experience.

### BRIEF DESCRIPTION OF THE DRAWINGS

FIG. 1 is a schematic diagram of the module relationship of the closed-loop artificial pancreas insulin infusion control system according to one embodiment of the present invention.

### DETAILED DESCRIPTION

As mentioned above, the artificial pancreas in the prior art cannot accurately sense the physical condition of the user and obtain total daily dose.

Studies have found that the reasons for the above problems are that the artificial pancreas in the prior art has insufficient algorithms, with single calculation method, for calculating the total daily dose, and has no structural components that can accurately sense user's physical activity.

In order to solve this problem, the present invention provides a closed-loop artificial pancreas insulin infusion control system which can automatically detect the physical condition of the user and accurately calculate the TDD value and the current insulin infusion dose, enhancing user experience.

Various exemplary embodiments of the present invention will now be described in detail with reference to the drawings. The relative arrangement of the components and the steps, numerical expressions and numerical values set forth in the embodiments are not to be construed as limiting the scope of the invention.

In addition, it should be understood that, for ease of description, the dimensions of the various components shown in the figures are not necessarily drawn in the actual scale relationship, for example, the thickness, width, length or distance of certain units may be exaggerated relative to other structures.

The following description of the exemplary embodiments is merely illustrative, and is not intended to be in any way limiting the invention and its application or use. The techniques, methods, and devices that are known to those of ordinary skill in the art may not be discussed in detail, but such techniques, methods, and devices should be considered as part of the specification.

It should be noted that similar reference numerals and letters indicate similar items in the following figures. Therefore, once an item is defined or illustrated in a drawing, it will not be discussed further in the following description of the drawings.

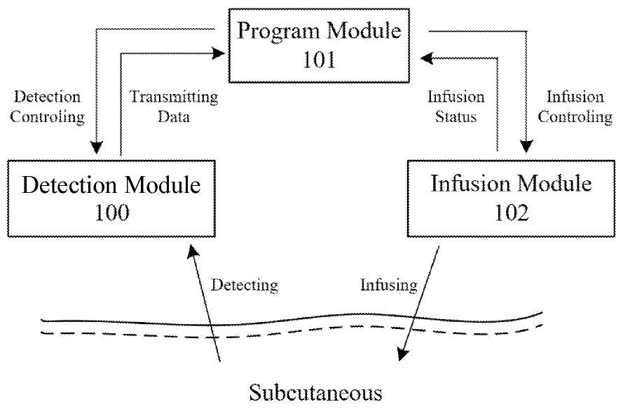

FIG.1 is a schematic diagram of the module relationship of the closed-loop artificial pancreas insulin infusion control system according to the embodiment of the present invention.

The closed-loop artificial pancreas insulin infusion control system disclosed in the embodiment of the present invention mainly includes a detection module 100, a program module 101, and an infusion module 102.

The detection module 100 is used to continuously detect the user's real-time blood glucose (BG) level. Generally, the detection module 100 is a Continuous Glucose Monitoring (CGM) for detecting real-time BG, monitoring BG changes, and also sending them to the program module 101.

The program module 101 is used to control the detection module 100 and the infusion module 102. Therefore, the program module 101 is connected to the detection module 100 and the infusion module 102, respectively.

Here, the connection refers to a conventional electrical connection or a wireless connection.

The infusion module 102 includes the essential mechanical structures used to infuse insulin and controlled by the program module 101. According to the current insulin infusion dose calculated by the program module 101, the infusion module 102 injects the currently insulin dose required into the user's body. At the same time, the real-time infusion status of the infusion module 102 can also be fed back to the program module 101.

The embodiment of the present invention does not limit the specific positions and connection relationships of the detection module 100, the program module 101 and the infusion module 102, as long as the aforementioned functional conditions can be satisfied.

As in an embodiment of the present invention, the three are electrically connected to form a single structure. Therefore, the three modules can be attached together on only one position of the user's skin. If the three modules are connected as a whole and attached in the only one position, the number of the device on the user skin will be reduced, thereby reducing the interference of more attached devices on user activities. At the same time, it also effectively solves the problem of the poor wireless communication between separating devices, further enhancing the user experience.

As in another embodiment of the present invention, the program module 101 and the infusion module 102 are electrically connected to each other to form a single structure while the detection module 100 is separately provided in another structure. At this time, the detection module 100 and the program module 101 transmit wireless signals to each other to realize mutual connection. Therefore, the program module 101 and the infusion module 102 can be attached on the same position of the user's skin while the detection module 100 is attached on the other position.

As in another embodiment of the present invention, the program module 101 and the detection module 100 are electrically connected to each other forming a single structure while the infusion module 102 is separately provided in another structure. The infusion module 102 and the program module 101 transmit wireless signals to each other to realize mutual connection. Therefore, the program module 101 and the detection module 100 can be attached on the same position of the user's skin while the infusion module 102 is attached on the other position.

As in another embodiment of the present invention, the three are respectively provided in different structures, thus being attached on different position. At this time, the program module 101, the detection module 100 and the infusion module 102 respectively transmit wireless signals to each other to realize mutual connection.

It should be noted that the program module 101 of the embodiment of the present invention also has functions such as storage, recording, and access to the database, thus, the program module 101 can be reused. In this way, not only can the user's physical condition data be stored, but also the production cost and the user's consumption cost can be saved. As described above, when the service life of the detection module 100 or the infusion module 102 expires, the program module 101 can be separated from the detection module 100, the infusion module 102, or both the detection module 100 and the infusion module 102.

Generally, the service lives of the detection module 100, the program module 101 and the infusion module 102 are different. Therefore, when the three are electrically connected to each other to form a single device, the three can also be separated from each other in pairs. For example, if one module expires firstly, the user can only replace this module and keep the other two modules continuous using.

Here, it should be noted that the program module 101 of the embodiment of the present invention may also include multiple sub-modules. According to the functions of the sub-modules, different sub-modules can be respectively assembled in different structure, which is not specific limitation herein, as long as the control conditions of the program module 101 can be satisfied.

In the embodiment of the present invention, the program module 101 is also used to obtain data including the insulin dose infused per day by users. Generally, for artificial pancreas, the current insulin dose required is closely related to the insulin dose infused per day by users in history. Preferably, in the embodiment of the present invention, the insulin dose infused per day by users includes the total amount of daily infusion dose data (d), or the bolus and basal data infused in different time periods, or the temporary basal data and the correction bolus data, or the infusion data after different events.

The program module 101 includes a manual input interface (not shown) or an automatic detection sub-module (not shown). By using the manual input interface or the automatic detection sub-module alone, or using the two combination, the program module 101 can obtain the user's physical condition data. This alone or combination using of these two modules enhances the flexibility in using the device.

For example, in an embodiment of the present invention, with the manual input interface, users can manually input the insulin dose infused per day by users into the program module 101 according to the clinical guidance. In another embodiment of the present invention, the program module 101 has already stored and recorded the user's previous insulin infusion data. With the automatic detection sub-module, the program module 101 can automatically obtain and calculate the insulin dose infused per day by users. Preferably, in the embodiment of the present invention, the user uses the manual input interface in combination with the automatic detection sub-module. At this time, the data automatically detected and the manually input can be combined and compared, making the program module 101 adjust the algorithm in real time for obtaining more accurate calculation outcome.

In other embodiments of the present invention, through the manual input interface, users can also input other information, such as meal information, exercise information, sleep information, and physical condition information into the program module 101, which is not specifically limited herein.

Generally, the purpose of using an artificial pancreas is to stabilize the BG level, that is, an appropriate dose of insulin needs to be infused into the user's body. However, the current insulin infusion dose is closely related to the total daily dose (TDD) which is an important factor influencing the current insulin infusion dose. Therefore, the program module 101 is imported into the total daily dose algorithm and the current insulin infusion algorithm, which are used to calculate the TDD and the current insulin infusion dose, respectively.

The current insulin infusion algorithm is used to calculate the current insulin infusion dose required. In the embodiment of the present invention, there are also many factors affecting the current insulin infusion dose, such as physical activity status, TDD, etc. Preferably, in the embodiment of the present invention, the TDD is one of the variable factors. Therefore, the more accurate the TDD or the more accurate the artificial pancreas sensing the user's activity status, the more accurate the current insulin infusion dose will be. And TDD can be obtained from calculating the aforementioned insulin dose infused per day by users according to the total daily dose algorithm. At the same time, the program module 101 can alone or in combination uses the detection data, the insulin dose infused per day by users and TDD data to calculate the current insulin infusion dose.

There are many factors that affect TDD, and some of them are related to the user's physical condition. Therefore, in the embodiment of the present invention, the variable factors of the total daily dose algorithm include one or more of the user's physical activity status, physiological status, psychological status, and meal status.

Here, the physiological status of the user includes one or more factors of weight, gender, age, disease condition, and menstrual period.

The user's psychological status includes emotional conditions such as anger, fear, depression, hyperactivity, and excitement.

The user's physical activity status includes general body stretching, exercise, or sleep. The control system can distinguish normal activities, exercise and sleep, making the control system more refined to control BG levels.

As mentioned above, TDD is obtained by the program module 101 by calculating the total amount of daily infusion dose data (d) in the previous two days or more according to the total daily dose algorithm. Preferably, in the embodiment of the present invention, TDD is obtained by the program module 101 by calculating the total amount of daily infusion dose data (d) in the previous seven days. Preferably, TDD is the average value of the total amount of daily infusion dose data (d).

In an embodiment of the present invention, if d₇, d₆, . . . , d₂, d₁ respectively represent the total amount of daily infusion dose data in the previous seventh day, the previous sixth day, . . . , the day before yesterday, and yesterday, then:

TDD=(d₇+d₆+ . . . +d₂+d₁)/7

that is, TDD is the arithmetic average of the total amount of daily infusion dose data (d).

If the time is much closer to the today, the total amount of daily infusion dose data (d) is much closer to the actual TDD. Therefore, in another embodiment of the present invention, different dₙ has different weights γₙ, such as the corresponding weights γ₇, γ₆, . . . , γ₂, γ₁, then:

TDD =γ₇d₇+γ₆d₆+ . . . +γ₂d₂+γ₁d₁

that is, TDD is the weighted average of the total amount of insulin infused per day (d).

It should be noted that the embodiment of the present invention does not limit the statistical method of dₙ. In yet another embodiment of the present invention, the TDD value can be determined by the median of the total amount of daily infusion dose data (d) in the previous seven days. In another embodiment of the present invention, the maximum value and minimum value of dₙ may be eliminated firstly, and then the averaging process is performed. Another embodiment of the present invention introduces variance or standard deviation method with discarding points with larger errors firstly and then performing averaging processing. In other embodiments of the present invention, a method of combining weighted average with a sliding data frame may also be used to make the calculation result of TDD more accurate.

Here, it should be noted that the sliding data frame refers to select the data, like from previous five consecutive days, as a data frame for statistics. And according to the passage of time, the data frame as a whole moves backward for several days, but still keeps including data of another previous five consecutive days. For the specific statistical method of the data in the sliding data frame, please refer to the foresaid, which will not be repeated herein.

As mentioned above, both TDD and the current insulin infusion dose are affected by physical activities. Therefore, the closed-loop artificial pancreas insulin infusion control system also includes a motion sensor (not shown) which is used to sense the user's physical activity. And the program module 101 can receive physical activity status information. The motion sensor can automatically and accurately sense the physical activity status of the user which will be sent to the program module 101, making the calculation result of the TDD or the current insulin infusion dose much more accurate, and enhancing the user experience. At the same time, providing the motion sensor in the module of the control system can improve the integration of the control system as much as possible, reduce the device size, and enhance the user experience.

The motion sensor is provided in the detection module 100, the program module 101 or the infusion module 102. Preferably, in the embodiment of the present invention, the motion sensor is provided in the program module 101.

It should be noted that the embodiment of the present invention does not limit the number of motion sensors and the installation positions of these multiple motion sensors, as long as the conditions for the motion sensor to sense the user's activity status can be satisfied.

The motion sensor includes a three-axis acceleration sensor or a gyroscope. The three-axis acceleration sensor or gyroscope can more accurately sense the body's activity intensity, activity mode or body posture, which ultimately makes the calculation result of the infusion more accurate. Preferably, in the embodiment of the present invention, the motion sensor is the combination of a three-axis acceleration sensor and a gyroscope.

In summary, the present invention discloses a closed-loop artificial pancreas insulin infusion control system which can automatically detect the physical condition of the user and accurately calculate the TDD value and the current insulin infusion dose, enhancing user experience.

While the invention has been described in detail with reference to the specific embodiments of the present invention, it should be understood that it will be appreciated by those skilled in the art that the above embodiments may be modified without departing from the scope and spirit of the invention. The scope of the invention is defined by the appended claims.

1. A closed-loop artificial pancreas insulin infusion control system, comprising:
a detection module configured to continuously detect a real-time blood glucose level;
a program module, connected to the detection module, is configured to obtain an insulin dose infused per day by users, and is also imported into a total daily dose algorithm and a current insulin infusion algorithm, wherein,
according to the insulin dose infused per day by the users, the total daily dose algorithm is used to calculate a total daily dose;
according to the blood glucose detected, the insulin dose infused per day by the users or the total daily dose, the current insulin infusion algorithm is used to calculate a current insulin infusion dose;
an infusion module, connected to and controlled by the program module, is configured to infuse an insulin required according to the data of the current insulin infusion dose; and
a motion sensor configured to sense a user's physical activity status which is sent to the program module and is one of variable factors of the total daily dose algorithm or the current insulin infusion algorithm.

2. The closed-loop artificial pancreas insulin infusion control system of claim 1, wherein
the program module includes a manual input interface or an automatic detection sub-module, and a method for the program module to obtain the insulin dose infused per day by the users includes:
the insulin dose infused per day by the users is manually input into the program module through the manual input interface; or
the insulin dose infused per day by the users is automatically detected, stored and calculated by the automatic detection sub-module.

3. The closed-loop artificial pancreas insulin infusion control system of claim 2, wherein
the insulin dose infused per day by the users includes a total amount of a daily infusion dose data, or a bolus and basal data infused in different time periods, or a temporary basal data and a correction bolus data, or an infusion data after different events.

4. The closed-loop artificial pancreas insulin infusion control system of claim 3, wherein
the total daily dose is obtained by calculating the total amount of the daily infusion dose data in previous two or more days according to the total daily dose algorithm, and the total daily dose is an average or a median of the insulin dose infused per day by the users, and the total daily dose is one variable factor of the current insulin infusion algorithm.

5. The closed-loop artificial pancreas insulin infusion control system of claim 4, wherein
the average includes an arithmetic average or a weighted average.

6. The closed-loop artificial pancreas insulin infusion control system of claim 3, wherein
the variable factors of the total daily dose algorithm include one or more of the user's physical activity status, a physiological status, a psychological status, and a meal status.

7. The closed-loop artificial pancreas insulin infusion control system of claim 6, wherein
the physiological status includes one or more factors of weight, gender, age, disease, and menstrual period.

8. The closed-loop artificial pancreas insulin infusion control system of claim 6, wherein
the physical activity status includes general body stretching, exercise, or sleep.

9. The closed-loop artificial pancreas insulin infusion control system of claim 6, wherein
a meal information, exercise information, sleep information, or physical condition information is manually input into the program module through the manual input interface.

10. The closed-loop artificial pancreas insulin infusion control system of claim 1, wherein
the motion sensor is provided in the detection module, the program module or the infusion module.

11. The closed-loop artificial pancreas insulin infusion control system of claim 10, wherein
the motion sensor includes a three-axis acceleration sensor or a gyroscope.

12. The closed-loop artificial pancreas insulin infusion control system of claim 1, wherein
any two of the detection module, the program module and the infusion module are connected to each other configured to form a single structure whose attached position on a shin is different from a third module.

13. The closed-loop artificial pancreas insulin infusion control system of claim 1, wherein
the detection module, the program module and the infusion module are connected together configured to form a single structure which is attached on only one position on a skin.
